(19) United States
(12) Patent Application Publication
(10) Pub. No.: US 2006/0079573 A1
(43) Pub. Date: Apr. 13, 2006

(54) Methods and compositions for preventing vasospasm

(76) Inventors: Jakob Vinten-Johansen, Grayson, GA (US); Faraz Kerendi, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/006,954

(22) Filed: Dec. 8, 2004

Related U.S. Application Data
(60) Provisional application No. 60/617,588, filed on Oct. 10, 2004.

(51) Int. Cl. A61K 31/366

(52) U.S. Cl. 514/453

(57) ABSTRACT

Methods and compositions for preventing or reducing vascular smooth muscle contraction are provided. Exemplary compositions comprise an MLCK inhibitor, for example wortmannin, in an amount effective to prevent or reduce vascular smooth muscle vasospams by about 90% for at least about 20 minutes. Methods for identifying other modulators of MLCK are also provided.

### CROSS-REFERENCE TO RELATED APPLICATIONS

This application claims priority to co-pending U.S. provisional application entitled, “Inhibition of Vasospasticity In Arterial Grafts And Other Vessels By Attenuation Of Myosin-Light Chain Kinase Activity,” having Ser. No. 60/617,588, filed Oct. 10, 2004, and which is incorporated by reference in its entirety.

### BACKGROUND

1. Technical Field

The present disclosure is generally related to methods and compositions for inhibition of vasospasm, in particular, methods and compositions for the inhibition and prevention of vascular smooth muscle contraction related to vascular grafts.

2. Related Art

Although recent reports have demonstrated favorable short and mid-term patency rates, early vasospasm of the radial artery when used as a conduit for coronary bypass surgery remains a clinically significant problem, particularly in patients who require the postoperative administration of vasopressors [Acar et al. (1992) Revival of the radial artery for coronary artery bypass grafting, Ann. Thorac. Surg., 54:652-60; Possati et al. (2003) Long-term results of the radial artery used for myocardial revascularization, Circulation, 108:1350-4; Tatoulis et al. (2002) The radial artery in coronary surgery: a 5-year experience-clinical and andiographic results, Ann. Thorac. Surg., 73:143-8]. A number of agents have been used for prophylaxis against arterial conduit vasospasm, including phosphodiesterase inhibitors, class I antiarrhythmic agents, alpha adrenergic receptor antagonists, calcium channel blockers, and nitric oxide donors. While these agents have been somewhat successful in alleviating intraoperative vasospasm, most are short-acting and provide for inhibition of vasospasm in the intraoperative period only. Furthermore, most of these antispasmodic therapies, which target specific stimulators of contraction, do not address the redundant extracellular stimuli and intracellular signals that regulate vascular smooth muscle cell contraction and arterial vasospasm. Although each of the above agents may inhibit one stimulus of vasospasm, other mechanisms remain operative that may cause vasospasm in the postoperative period.

Accordingly, there is a need for additional compositions and methods for treating or preventing vasospasms.

### SUMMARY

Aspects of the present disclosure provide methods and compositions for inhibition, prevention, or reduction of vasospasm, in particular contraction of vascular smooth muscle. The compositions are useful for the prevention or treatment of vasospasms, for example vasospasms related to vascular graft procedures, including but not limited to arterial graft procedures. It has been discovered that inhibition of myosin light chain kinase (MLCK) with inhibitors including, but not limited to wortmannin or derivatives thereof, can prevent postoperative vasospasm for durations of at least about 20 minutes, typically for at least about 1 to about 2 hours. In some aspects, inhibition of MLCK activity occurs either directly (e.g., via wortmannin or other MLCK inhibitors) or by inhibiting regulators such as rho-rho-kinase pathway ( for example by Y27632) and/or promoting myosin light chain phosphorylase activity (also called phosphatase), which removes the phosphate group from MLC and inhibits contraction (see FIGS. 1 and 2).

One aspect provides a method for preventing or reducing vasospasms in a host comprising administering to the host an amount of an MLCK inhibitor sufficient to prevent vasospasms. A representative MLCK inhibitor includes, but is not limited to wortmannin, derivatives thereof, and prodrugs thereof.

Another aspect provides a composition comprising an inhibitor of MLCK, typically an irreversible inhibitor of MLCK, in an amount effective to reduce contractile responses of vascular smooth muscle in a host by about 90% for at least about 20 minutes, typically about 1 to about 2 hours post administration, or by about 50% for at least about 1 to about 48 hours post administration. The composition can be a pharmaceutical composition optionally containing a pharmaceutically acceptable carrier, excipient, or controlled release means. In some aspects, the composition is administered non-systemically.

Another aspect provides a method for identifying compounds for preventing or reducing vasospasms comprising contacting vascular smooth muscle cells, for example vascular tissue, with a test compound, assaying the vascular smooth muscle cells for contraction and MLCK activity, and selecting the compound that inhibits MLCK activity and/or inhibits rho-rho kinase pathway individually or in combination and reduces or prevents vascular tissue contraction. The method optionally includes determining the ability of the test compound to induce apoptosis and selecting the test compound that does not induce apoptosis or induces apoptosis to a less extent than wortmannin. In still another aspect, a test compound is selected that irreversibly inhibits MLCK and has less than about 50% to less than about 10% inhibition of another enzyme, for example, inhibits PI-3 kinase by less than about 10%.

Yet another aspect provides a method for preventing vasospasm in host comprising administering an irreversible inhibitor of MLCK to a blood vessel at a site of a graft in an amount sufficient to inhibit or reduce contraction of the blood vessel at the site of the graft.

### BRIEF DESCRIPTION OF THE FIGURES

Many aspects of the disclosure can be better understood with reference to the following figures. The components in the figures are not necessarily to scale, emphasis instead being placed upon clearly illustrating the principles of the present disclosure. Moreover, in the figures, like reference numerals designate corresponding parts throughout the several views.

FIGS. 1A-1D are bar graphs showing the force of contraction in response to 1 μM NE (A), 1 μM 5-HT (B), 3 μM U46619 (C), and 60 mM KCl (D) at 2 and 48 h after soaking in 1 mM WT (open bars) or control buffer (shaded bars). WT-treated vessels contracted significantly less at both the 2 and 48 h time point. (*Indicates P<0.001 vs. 2 h control group. # indicates P<0.001 vs. 48 h control group).

FIG. 2 is a line graph showing the relaxation of control vessels (open squares) and WT-treated vessels (shaded triangles) in response to increasing concentrations of sodium nitroprusside (SNP) after pre-contraction with 3 μM U46619. While control vessels relaxed to a greater extent at lower concentrations of SNP, there was no difference between groups at higher concentrations (* indicates P<0.05; # indicates P=not significant).

FIGS. 3A and 3B are photomicrographs (hematoxylin and eosin) of control vessels (A) and WT-treated vessels (B) at 400× magnification. Note the greater extent of apoptotic nuclei (black arrows) stained red by the TUNEL method in the smooth muscle layer of control vessels (A) as compared to WT-treated vessels (B).

FIGS. 4A and 4B are photomicrographs (hematoxylin and eosin) of control vessels (A) and WT-treated vessels(B) at 400× magnification. Note the greater extent of apoptotic nuclei (black arrows) stained red by the TUNEL method in the endothelium of control vessels (A) as compared to WT-treated vessels (B).

### DETAILED DESCRIPTION

In accordance with the purposes(s) of the present disclosure, as embodied and broadly described herein, embodiments of the present disclosure, in one aspect, relate to methods and compositions for the inhibition, reduction, or prevention of vascular smooth muscle contraction by inhibiting or interfering with the myosin light chain kinase (MLCK) activity.

Prior to describing the various embodiments, the following definitions are provided and should be used unless otherwise indicated.

Definitions:

The term “organism” or “host” refers to any living entity comprised of at least one cell. A living organism can be as simple as, for example, a single eukaryotic cell or as complex as a mammal, including a human being.

The term “vasospasm” refers to a narrowing of an artery which bring blood to your brain, heart, and/or other organs. Vasospasm may occur in all of the parts of the body at the same time or in only certain parts of your body at specific times.

The term “graft” refers to a transplanted organ or tissue.

The term “vessel” refers to an elastic tubular channel, such as an artery, a vein, or a capillary, through which the blood circulates.

The term “myosin light chain kinase” refers to an enzyme that binds both to actin and myosin II. The phosphorylation of the 20 kDa regulatory light chains of MLCK regulates the contractile interaction between actin microfilaments and conventional smooth muscle and non-muscle myosin II.

As used herein, “wortmannin” refers to a fungal metabolite that specifically inhibits phosphatidylinositol 3-kinase (PI-3), mitogen-activated protein kinase (MAPK) and myosin light-chain kinase (MLCK). Wortmannin has a molecular weight of about 428.4. Wortmannin is obtained from Penicillium fumiculosum. It is hygroscopic, should be protected from light and moisture, and appears as an off-white to pale yellow solid.

The term “analog” refers to a chemical compound that is structurally similar to another but differs slightly in composition (as in the replacement of one atom by an atom of a different element or in the presence of a particular functional group) The term “derivative” refers to a chemical substance related structurally to another substance and theoretically derivable from it. A derivative can also be a substance that can be made from another substance.

A “pharmaceutical composition” refers to a mixture of one or more of the compounds described herein, or pharmaceutically acceptable salts thereof, with other chemical components, such as physiologically acceptable carriers and excipients. The purpose of a pharmaceutical composition is to facilitate administration of a compound to an organism.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

As used herein, a “pharmaceutically acceptable carrier” refers to a carrier or diluent that does not cause significant irritation to an organism and does not abrogate the biological activity and properties of the administered compound. This term encompasses any of the standard pharmaceutical carriers, such as a phosphate buffered saline solution, water and emulsions such as an oil/water or water/oil emulsion, and various types of wetting agents, including solvents such as alcohols and DMSO.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. See, e.g., Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs--principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “treating” includes alleviating the symptoms associated with a specific disorder or condition and/or preventing or eliminating said symptoms.

Having defined some of the terms herein, the various embodiments of the disclosure will be described.

Inhibitors of MLCK

One embodiment of the present disclosure provides compositions for the treatment, reduction, or inhibition of vascular smooth muscle contraction. An exemplary composition comprises an MLCK inhibitor in an amount effective to inhibit MLCK activity in vascular tissue. The MLCK inhibitor can be a reversible or irreversible inhibitor and typically is in an amount effective to inhibit MLCK activity for at least about 20 minutes post administration. Another embodiment provides a composition comprising an amount of an MLCK inhibitor effective to inhibit MLCK of vascular smooth muscle cells by at least about 1 to about 2 hours post administration. Still another embodiment provides a composition comprising an MLCK inhibitor in an amount sufficient to inhibit MLCK of smooth muscle cells by at least about 50% for at least about 1 hour, typically for at least about 48 hours post administration. The compositions optionally include a pharmaceutically acceptable carrier or excipient. In certain embodiments, the compositions are administered locally to smooth muscle cells or tissues, and non-systemically.

In some embodiments, the disclosed compositions prevent or inhibit the phosphorylation of the 20 kD subunit of myosin light chain (MLC) in vascular smooth muscle cells or tissues. The phosphorylation of the 20 kD subunit of myosin light chain (MLC) by myosin light chain kinase (MLCK) is a central regulatory step in the development of force in the vascular smooth muscle cell. Phosphorylation of MLC by MLCK allows binding of actin to myosin, followed by activation of the myosin-ATPase, and the subsequent contraction of the smooth muscle cell [Somlyo and Somlyo (1994) Signal transduction and regulation in smooth muscle, Nature, 372:231-6]. Wortmannin (WT), a product of the fungus Talaromyces wortmannin, is an irreversible inhibitor of MLCK [Nakanishi et al. (1992) Wortmannin, a microbial product inhibitor of myosin light chain kinase, J. Biol. Chem., 267:2157-63]. Wortmannin was first introduced as an inhibitor of MLCK in 1992 [Nakanishi et al., 1992].

In another embodiment of the disclosure, regulators of MLCK activity such as rho-rho kinase pathway are inhibited. An exemplary inhibitor is Y27632. The rho-rho kinase pathway directly promotes phosphorylation and activation of MLCK, and directly lifts the inhibitory effect of myosin light chain phosphorylase on MLCK thereby promoting contraction. Therefore, inhibition of the regulators of MLCK such as rho-rho kinase pathway would also result in inhibition of smooth muscle contraction via the MLCK pathway.

Although the present disclosure exemplifies the use of wortmannin for the inhibition of MLCK in vascular smooth muscle cells, it is understood that the disclosed methods and compositions are not limited to wortmannin. Wortmannin is known in the art, and the biosynthetic production of wortmannin and the analogs or derivatives thereof is also known in the art. Accordingly, the disclosure encompasses the use of derivatives of wortmannin, in particular derivatives that do have reduced or no undesirable effects of wortmannin including, but not limited to proapoptotic effects. Exemplary derivatives of wortmannin include, but are not limited to 11-desacetyl-11-(1-iodoacetyl)-wortmannin, 11-desacetyl-11-(1-iodoacetyl)-wortmannin, 17-dihydro-17-(1-iodoacetyl)-wortmannin, or combinations thereof. The inhibitor of MLCK can include water soluble drug polymers comprising wortmannin or a derivative thereof including, but not limited to polyethylene glycol polymers.

The synthesis of wortmannin and derivatives thereof is described in U.S. Pat. No. 5,480,906, which is incorporated herein by reference in its entirety. Generally, wortmannin is produced by the fermentation of any one of a number of previously disclosed microorganisms such as Talaromyces wortmannin and Penicillium wortmannin, Myrothecium roridium, and Fusarium. Following fermentation, wortmannin is extracted and purified via known methods. Preferably, wortmannin is microbially synthesized and isolated in substantially pure form from a fermentation culture (one such fermentation culture is identified as A24603.1).

Certain embodiments of the present disclosure use wortmannin, a product of the fungus Talaromyces wortmannin, as a control compound in assays for identifying additional inhibitors of MLCK. In such assays, the activity of a test compound can be compared to the activity of wortmannin. For example, test compounds can be selected that have similar or optionally more potent inhibitory effects on MLCK than wortmannin. Moreover, compounds can be selected that do not exhibit undesirable activities of wortmannin, including but not limited to inducing apoptosis.

Relative to the present disclosure, it has been discovered that wortmannin is also a potent inhibitor of vasoconstriction in the intact canine radial and the porcine gastroepiploic arteries. Furthermore, the data indicate that the inhibitory effect is long lasting compared to other known treatments for vasospasm. In certain embodiments, the inhibition of MLCK can persist for at least 20 minutes to about 48 hours.

In another embodiment, compositions comprising an MLCK inhibitor optionally include a second active ingredient. Representative second active ingredients include, but are not limited to anti-inflammatory agents, for example non-steroid anti-inflammatory compounds (adenosine and adenosine analogs or adenosine regulating agents, nitric oxide and nitric oxide donors), antioxidants, lipids, HMGCO-A reductase inhibitors, antibiotics, analgesic compounds, blood thinners, or combinations thereof. Exemplary non-steroid anti-inflammatory compounds include, but are not limited to COX-2 inhibitors, salicylates, diflunisal, ibuprofen, ketoprofen, nabumetone, piroxicam, naproxen, diclofenac, indomethacin, sulindac, tolmetin, etodolac, ketorolac, oxaprozin, celecoxib, and combinations thereof. The second active ingredient can optionally be administered in combination with the disclosed compositions in separate formulations. The second active ingredient can inhibit or reduce the expression of inflammatory cytokines including, but not limited to TNF-α, IL-1β, or both.

The second active ingredient can also be a second inhibitor of MLCK. Additional MLCK inhibitors include, but are not limited to H—RKKYKYRRK—NH₂ (SEQ ID NO: 1), MLCK Inhibitor Peptide 18, ML-7 and KT5926 [Katoh, K. et al. J. Physiol Cell Physiol. 2001 June;280(6):C1669-79], N-(2-aminoethyl)-5-chloronaphthalene-1-sulfonamide, UCN-1028C, (±)-1-(5-isoquinolinesulfonyl)-2-methylpiperazine, N-[2-(methylamino)ethyl]-5-isoquinolinesulfonamide, N-[2-(p-bromocinnamylamino)ethyl]-5-isoquinolinesulfonamide, 1-(5-chlornaphthalene-1-sulfonyl)-1H-hexahydro-1,4-diazepine, 1-(5-isoquinolinylsulfonyl)piperazine, 1-(5-lsoquinolinesulfonyl)homopiperazine, and combinations thereof.

Still another embodiment provides a composition comprising an MLCK inhibitor in an amount effective to prevent calcium dependent vascular smooth muscle cell contraction in a host. Calcium dependent contraction can be triggered, for example, by norepinephrine, angiotensin II, vasopressin, endothelin-1, and/or thromboxane A₂ when these ligands bind to their respective receptors on the vascular smooth muscle cell. The signal transduction cascade that results when these ligands bind their cognate receptors triggers the release of calcium from the sarcoplasmic reticulum and activate voltage-gated calcium channels (VG) in the cell membrane, which then allow entry of extracellular calcium into the cell. This rise in intracellular calcium concentration is associated with the binding of calcium to calmodulin, forming the calcium-calmodulin (Ca—CaM) complex, thereby stimulating activation of MLCK. Once phosphorylated (i.e. activated), myosin is able to interact with actin, thereby forming the actin-myosin cross bridge, after which cross-bridge cycling ensues and smooth muscle cell contraction occurs. In the deactivated state or unphosphorylated state, myosin is unable to interact with actin, which eventually leads to smooth muscle cell relaxation.

In other embodiments, the disclosed compositions inhibit or reduce calcium independent vascular smooth muscle contraction. An exemplary calcium independent pathway for vascular smooth muscle contraction is the rho-rho kinase pathway. Although the intracellular events leading to smooth muscle contraction have classically been associated with the rise of intracellular calcium, there is recent evidence that a rise in intracellular calcium is not an absolute requirement. This phenomenon that SMC contraction occurs without a rise in intracellular calcium has been termed calcium sensitization, implying that the cell becomes more sensitive to calcium, and contraction occurs despite minimal changes in intracellular calcium concentration [Fukata et al. (2001) Rho-rho-kinase pathway in smooth muscle contraction and cytoskeletal reorganization of non-muscle cells, Trends Pharm. Sci., 22(1):32-39; Somlyo and Somlyo, 1994]. The GTPase Rho plays an important role in the process of calcium sensitization. Rho is activated via G protein-coupled receptors when a contractile agonist binds to its receptor on the cell surface. Once activated, Rho in turn activates Rho-kinase, which subsequently phosphorylates and inhibits the myosin binding subunit of myosin phosphatase. With myosin phosphatase inhibited, the opposing reaction of myosin light chain phosphorylation by the basal activity of MLCK is favored, causing sustained contraction with little change in intracellular calcium concentration (Fukata et al., 2001). The disclosed compositions can inhibit calcium independent smooth muscle contraction by inhibiting MLCK so that basal MLCK activity is insufficient to induce contraction.

Still another embodiment provides a medical device, for example a stent, impregnated or coated with MLCK inhibitor. Vascular stents are known in the art and can be made of an expandable mesh material configured to be inserted into a blood vessel or conduit. The MLCK inhibitor can be formulated to diffuse from the stent in a predetermined amount. The MLCK inhibitor or rho kinase inhibitor may also be introduced using catheters of various configurations including single balloon catheters and double balloon catheters. In the latter case, the inhibitor or regulator of MLCK activity can be infused in the space bounded by the two balloons (balloon interspace) and allowed to “dwell” there for a period of time, ranging from about 10 seconds to about 10 minutes.

Screening

The disclosure also provides methods for identifying modulators of vascular muscle contraction including, but not limited to MLCK inhibitors. As used herein the term “test compound” or “modulator” refers to any molecule that may potentially inhibit vascular muscle contraction. Some test compounds and modulators can be compounds that are structurally related to wortmannin and prodrugs or derivatives of wortmannin. Using lead compounds to help develop improved compounds is known as “rational drug design” and includes not only comparisons with known inhibitors and modulators, but predictions relating to the structure of target molecules.

One embodiment provides a method for identifying inhibitors of myosin light chain phosphorylation including, but not limited to, an inhibitor of myosin light chain kinase (MLCK), and selecting the test compound that interferes with myosin light chain kinase compared to a control compound. A representative control compound includes, but is not limited to wortmannin. The test compounds can be further screened for compounds that have less or none of the undesirable effects of wortmannin. A representative undesirable effect of wortmannin includes the induction of apoptosis. Accordingly, test compounds can be selected that inhibit MLCK and do not induce apoptosis, or induce apoptosis to a lesser extent than wortmannin.

Test compounds can also be screened for the ability to modulate the activity of enzymes other than MLCK. Generally, compounds that do not modulate enzymes other than MLCK are selected. In one embodiment test compounds that inhibit MLCK and inhibit phosphatidylinositol-3 (PI-3) kinase by less than about 10% are selected. The ability of the test compounds to induce apoptosis, necrosis of treated tissues or cells, inflammation, and/or coagulation can be determined, and test compounds that induce these effects by less than about 10% as compared to control compounds can be selected.

Generally, test compounds can be selected that inhibit vascular smooth muscle contraction by about 90% for at least 20 minutes, typically for at least 1 hour, more typically for about 2 hours. In other embodiments, test compounds can be selected that inhibit vascular smooth muscle contraction by about 50% for at least about 48 hours, typically for at least about 24 hours, more typically for at least about 1 to about 12 hours.

In another embodiment, small molecule libraries that are believed to meet the basic criteria for useful drugs can be screened to identify useful compounds. Screening of such libraries, including combinatorially generated libraries (e.g., expression libraries), is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds.

Test compounds may include fragments or parts of naturally-occurring compounds for example wortmannin, or may be found as active combinations of known compounds, which are otherwise inactive. Compounds isolated from natural sources, such as animals, bacteria, fungi, plant sources, including leaves and bark, and marine samples can be assayed as candidates for the presence of potentially useful pharmaceutical agents. It will be understood that the pharmaceutical agents to be screened could also be derived or synthesized from chemical compositions or man-made compounds. Thus, it is understood that the test compound identified by embodiments of the present disclosure may be peptide, polypeptide, polynucleotide, small molecule inhibitors, small molecule inducers, organic or inorganic, or any other compounds that may be designed based on known inhibitors.

In addition to the test compounds initially identified, other sterically similar compounds may be formulated to mimic the key portions of the structure of the test compounds or modulators.

An inhibitor according to the present disclosure may be one which exerts its inhibitory effect upstream, downstream, directly, or indirectly on myosin light chain kinase (MLCK) or on regulators of MLCK activity.

In some embodiments, the assays can include random screening of large libraries of test compounds. Alternatively, the assays may be used to focus on particular classes of compounds suspected of modulating the function or expression of MLCK.

In Vitro Assays

Another embodiment provides for in vitro assays for the identification of vascular smooth muscle contraction inhibitors, such as but not limited to MLCK inhibitors, including but not limited to wortmannin and prodrugs or derivatives of wortmannin. Such assays generally use isolated molecules and can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule, for example MLCK in a specific fashion is strong evidence of a related biological effect. Such a molecule can bind to MLCK and sterically inhibit MLCK. The binding of a molecule to a target may, in and of itself, be inhibitory, due to steric, allosteric or charge-charge interactions or may downregulate or inactivate MLCK. The target may be either free in solution, fixed to a support, or expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

Cell Assays

Other embodiments include methods of screening compounds for their ability to modulate and downregulate MLCK in cells. Various cell lines can be utilized for such screening assays, including cells specifically engineered for this purpose. Suitable cells include, but are not limited to, mammalian cells. Cells can also be engineered to express MLCK or a modulator of MLCK or a combination of both MLCK or a modulator of MLCK. Furthermore, those of skill in the art will appreciate that stable or transient transfections, which are well known and used in the art, may be used in the disclosed embodiments. Cells in which MLCK and regulators have been overexpressed, suppressed or knocked out can also be used.

Depending on the assay, cell culture may be required. The cell is examined using any of a number of different physiologic assays. Alternatively, molecular analysis may be performed, for example, looking at protein expression, mRNA expression (including differential display of whole cell or polyA RNA) and others.

In Vivo Assays

In vivo assays involve the use of various animal models, including non-human transgenic animals that have been engineered to have specific defects, or carry markers that can be used to measure the ability of a test compound to reach and affect different cells within the organism. Suitable animals include mice, rats, rabbits, hamsters, guinea pigs, gerbils, woodchucks, cats, dogs, sheep, goats, pigs, cows, horses and monkeys (including chimps, gibbons and baboons).

Assays for MLCK inhibitors may be conducted using an animal model derived from any of these species. In such assays, one or more test compounds are administered to an animal, and the ability of the test compound(s) to alter one or more characteristics, as compared to a similar animal not treated with the test compound(s), identifies a modulator. The characteristics may be any of those discussed above with regard to the function of a particular compound (e.g., enzyme, receptor, hormone) or cell (e.g., growth or regeneration).

Methods

One embodiment provides a method for treating vasospasms, for example vascular smooth muscle contraction, in host. The disclosed compositions can be used to inhibit, prevent, reduce, or treat vasospams, in particular vasospasms related to bypass surgery or coronary artery or other vessel bypass graft. This may apply to other surgeries than coronary artery (open heart) surgeries in which arterial conduits may be placed, for example (but not limited to) femoral artery-popliteal artery bypass, brachial artery bypass, carotid artery bypass. Compositions containing one or more MLCK inhibitors can be topically or directly applied to vascular smooth muscle cells prior to surgery, during surgery, or after surgery. The MLCK inhibitors and/or regulators such as myosin light chain kinase phosphatase can be modified to prevent or reduce diffusion away from the site of application. For example, the MLCK inhibitor can be reversibly conjugated to a polymer such as polyethylene glycol, cellulose, or inert polymer. Modifications may also include changing the electronic or ionic charges or their distribution on the molecule to enhance movement into cells and tissues by e.g. electrophoretic techniques.

Pharmaceutical Compositions

Embodiments of the disclosure include pharmaceutical compositions comprising an MLCK inhibitor or regulators of MLCK such as rho-rho kinase pathway components, prodrug, pharmaceutically acceptable salt, polymorph, solvate, hydrate, dehydrate, co-crystal, anhydrous, or amorphous form thereof.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. A parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that include primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of one or more MLCK inhibitors or MLCK regulators such as rho-rho kinase, myosin light chain kinase phosphatase promoters in a dosage form may differ depending on factors such as, but not limited to, the route by which it is to be administered to patients.

Additionally, the compounds and/or compositions can be delivered using lipid- or polymer-based nanoparticles. For example, the nanoparticles can be designed to improve the pharmacological and therapeutic properties of drugs administered parenterally [Allen, T. M., Cullis, P. R. Drug delivery systems: entering the mainstream. Science. 303(5665):1818-22 (2004)]. Nano particles may also include liposomal encapsulation formulae and ultrasound-dispersible or disruptible carriers.

Topical dosage forms of the disclosure include, but are not limited to, creams, lotions, ointments, gels, sprays, aerosols, solutions, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms including a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th Ed., Mack Publishing, Easton, Pa. (1990). Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Additional transdermal dosage forms include “reservoir type” or “matrix type” patches, which can be applied to the skin and worn for a specific period of time to permit the penetration of a desired amount of active ingredient. Examples of transdermal dosage forms and methods of administration that can be used to administer the active ingredient(s) of the disclosure include, but are not limited to, those disclosed in U.S. Pat. Nos.: 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable. Depending on the specific tissue to be treated, additional components may be used prior to, in conjunction with, or subsequent to treatment with pharmaceutically acceptable compounds or compositions of a MLCK inhibitor of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate). The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent.

Experimental Procedures

Materials

Wortmannin (WT), Krebs-Henseleit (KH) buffer, nor-epinephrine (NE), serotonin (5HT), KCl, U46619 (U46), and sodium nitroprasside (SNP) were purchased from Sigma (St Louis, Mich.). All drugs were dissolved in water except WT which was dissolved in dimethyl sulfoxide (DMSO) and U46 which was dissolved in ethanol.

Animal Preparation and Artery Harvest

All animal care was conducted under the approval of the institutional animal care and use committee, and in compliance with the European Convention on Animal Care.

Canine brachial arteries, equivalents of radial arteries in humans, were used in this study since they were more readily available and we could obtain longer segments than human radial arteries. Based on previous publications, it has been determined that the contractile and relaxation properties of these canine vessels are similar to the properties of human radial arteries in organ chamber studies [Corvera et al. (2003) Pretreatment with phenoxybenzamine attenuates the radial artery's vasoconstrictor response to α-adrenergic stimuli, J. Thorac. Cardiovasc. Surg., 126:1549-54; Velez et al., 2001]. The arteries were harvested as a pedicle graft and stored in 4° C. KH buffer (in mM/1: glucose 11; magnesium sulfate, 1.2; potassium phosphate 1.2; KCl, 4.7; sodium chloride 118; calcium chloride, 2.5) at pH 7.4 until ready for use.

Vessel Preparation, Treatment and Testing of Contractile Function

The vessels were carefully skeletonized, cut into 4-5 mm segments, and soaked in a 1 mM solution of WT in KH buffer and 10% DMSO (used to increase solubility of WT), pH 7.4 at 37° C. for 60 min. Control vessels were soaked in KH buffer with 10% DMSO only. The vessels were then rinsed well with KH to remove all unbound drug. For testing of contractile responses, the arterial segments were mounted on steel hooks in glass organ chambers (Radnoti Glass, Monrovia, Calif.) in KH buffer at pH 7.4 and 37° C., and continuously bubbled with a gas mixture of 95% O₂ and 5% CO₂. One steel hook was held stationary while the other was attached to a force transducer. Force generated by the vessels in response to various agonists was recorded using an analog-to-digital converter and SPECTRUM Cardiovascular Acquisition and Analysis software (Wake Forest University, Winston-Salem, N.C.). The vessel segments were allowed to stabilize at a baseline tension of 3 g for 60 min prior to testing the effect of various vasoconstrictor agents. During this time, the buffer was changed every 20 min. The total time from the end of WT treatment until contractile responses were tested was approximately 2 h. The contractile responses to 1 μM NE (α1 receptor-dependent, calcium-dependent), 1 μM 5-HT (serotonin receptor-dependent, calcium-independent), 3 μM U46 (thromboxane A2 receptor-dependent, calcium-independent), and 60 mM KCl (receptor-independent depolarizing agent) were then quantified. These drug concentrations were chosen after preliminary studies indicated that they would provide a level of contraction that was detectable by the described system. The order in which the constricting agents were administered was varied randomly in order to avoid the effects of one drug on another. However, KCl was always administered last since high concentrations of KCl have been shown to have damaging effects on vascular endothelium. The vessels were thoroughly washed and allowed to stabilize for 20 min between vasoconstrictor agents. The resting tension was adjusted to 3 g during each stabilization period.

Contractile Response at 48 h

To test the duration of MLCK inhibition by the 60 min pre-treatment with 1 mM WT, a subset of vessels were harvested and treated with WT as above, then incubated in drug-free sterile culture medium [Dulbecco's modified eagle medium (Sigma, St Louis Mo.), with 10% newborn calf serum (Gibco, Inc.) and 1% penicillin-streptomycin] under sterile conditions for 48 h in an incubator aerated with 95% O₂ and 5% CO₂. The culture medium was changed every 12 h during this time. At the end of the 48 h period, the vessels were removed, rinsed with KH buffer, and contractile responses were tested as described above.

Testing of Smooth Muscle Relaxing Capacity

Vascular smooth muscle integrity has been shown to be impaired after some treatment strategies [Gao et al. (2003) Detrimental effects of papaverine on the human internal thoracic artery, J. Thorac. Cardiovasc. Surg., 126:179-85; van Son et al. (1992) Detrimental sequelae on the wall of the internal mammary artery caused by hydrostatic dilation with diluted papaverine solution, J. Thorac. Cardiovasc. Surg., 104:972-6]. Therefore, to test the vasorelaxation capacity of the vessels after treatment with WT, the vessels were pre-constricted with 3 μM U46, and then exposed to incrementally increasing concentrations (50 nM to 5 mM) of SNP, a direct smooth muscle dilator.

Endothelial-dependent vasorelaxation (i.e., vasodilatation in response to acetylcholine or bradykinin) was not tested in this study since WT is a known inhibitor of phosphoinosi-tide-3 (PI-3) kinase, which mediates acetylcholine- and bradykinin-induced activation of endothelial nitric oxide synthase (eNOS) [Kitayama et al.(2000) role of phosphatidylinositol 3-kinase in acetylcholine-induced dilation of rat basilar artery, Stroke, 31:2487-93].

Evaluation of Vessels for Mornhologic Injury and Apoptosis

Prolonged storage in cell culture environments has the potential to cause cellular injury, which might impair smooth muscle function. To confirm that the lack of the observed contraction in WT-treated vessels was not a function of cellular damage, a subset of vessels was tested for histological signs of injury and apoptosis after treatment with WT and incubation for 48 h. For histology, formalin-fixed vessels were embedded in paraffin blocks, cut into sections for slide preparation, and stained with hematoxylin and eosin (H and E) prior to examination for signs of morphologic damage.

Endothelial and smooth muscle cell apoptosis was evaluated using the terminal deoxynucleotidyl transferase (TdT)-mediated dUTP nick end labeling (TUNEL) method. In preparation for the TUNEL assay, the vessels were embedded in optimal cutting temperature compound (OCT, Sakura Finetek, Torrance, Calif.). Cryosections from frozen tissue were obtained using a Hacker-Bright cryostat and thaw-mounted onto Fisher-Plus slides (Fisher Scientific). Determination of apoptosis was performed using an in situ cell death detection kit (Roche Applied Science, Indianapolis, Ind.). DNA strand breaks were labeled with fluorescein-dUTP, followed by the addition of a secondary, antifluorescein antibody conjugated with alkaline phosphatase, a converter which generates a red color from Vector Red substrate. Folio wing counterstaining with hematoxylin and dehydration in graded alcohols, the number of apoptotic cells (indicated by red-stained nuclei) per high power field (HPF) was counted under light microscopy and is expressed as a percentage of the total number of nuclei per HPF.

Statistical Analysis

All data are expressed as mean±standard error of the mean (SEM). Differences in groups were determined by a one way analysis of variance (ANOVA) with Student-Newman-Keuls post-hoc analysis corrected for multiple comparisons. If data failed tests of normality, a Kruskal-Wallis ANOVA on ranks or a Mann-Whitney Rank Sum test was performed. A P-value of less than 0.05 was considered to be statistically significant.

### EXAMPLES

### Example 1

### Inhibition of Vasoconstriction by Blockade of MLCK Activity

In this example, a myosin light chain kinase inhibiting (MLCK) agent, wortmannin, is used to inhibit or bind to MLCK to attenuate radial artery vasoconstriction up to 48 hours after brief treatment. This strategy may prevent vasospasm of arterial grafts from all causes for several postoperative days.

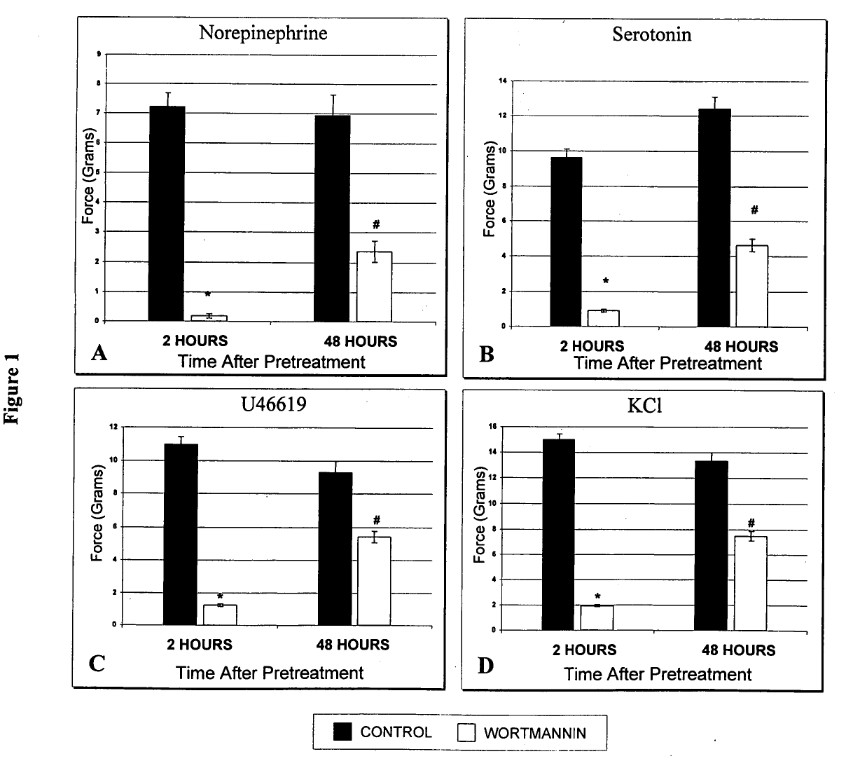

Two hours after treatment, WT-treated vessels contracted significantly less than control vessels in response to NE (0.19±0.07 vs. 7.22±0.37 g, P<0.001); 5HT (0.92±0.35 vs. 9.64±0.67 g, P<0.001; U46 (1.25±0.17 vs. 10.99±0.50 g, P<0.001) and KCl (1.98±0.27 vs. 15.00±0.48 g, P<0.001) (FIGS. 1-4, n=54 segments from 12 animals in control group and 28 segments from six animals in WT group). After 48 h of incubation, WT-treated vessels regained some contractility. However, the magnitude of contraction in WT-treated vessels was still significantly reduced in response to all vasoconstricting agents when compared to time-matched controls: 2.36±0.17, vs. 6.95±0.47 g for NE (P<0.001); 4.67±0.39 vs. 12.42±0.70 g for 5HT (P<0.001); 5.42±0.34 vs. 9.29±0.74 g for U46 (P=0.008); 7.49±0.48 vs. 13.32±0.60 g for KCl (P<0.001) (FIGS. 1A-D, n=35 segments from five animals in each group). There was no significant difference in the magnitude of contractile force in control vessels between 2 and 48 h.

### Example 2

### Endothelial-Independent Smooth Muscle Relaxation

The capacity of the vascular smooth muscle to relax independently of the endothelium was evaluated by pre-constricting vessels with U46, followed by exposure to incrementally increasing concentrations of SNP, a direct smooth muscle relaxing agent. WT-treated vessels were not tested at the 2 h time point since they do not contract sufficiently (maximal contraction=1.25±0.17 g vs. 10.99±0.50 g for controls) in the early period in order to pre-constrict and subsequently relax by SNP; they were, however, tested at the 48 h time point. Two hours after soaking, control vessels relaxed 99.04±7.30% from their pre-constricted state in response to SNP.

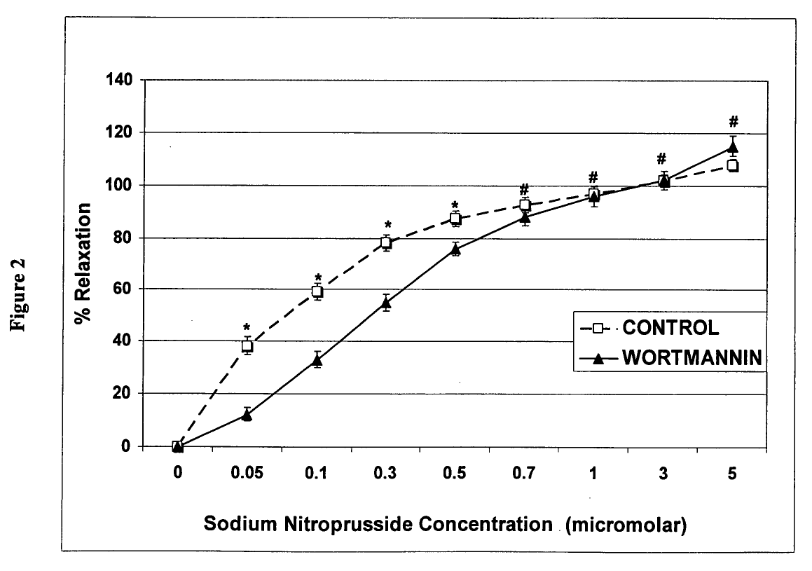

At 48 h, with lower concentrations of SNP L≦0.5 μM), the WT-treated group had a diminished relaxation response compared to controls (76.01±2.60% for WT-treated vs. 87.49±2.91% for controls, P=0.01, FIG. 2).

### Example 3

### Histologic Evaluation of Vessels

The vessels were tested for microscopic evidence of injury 48 h after treatment and incubation. Control vessels did not show any signs of morphologic damage by hematoxylin and eosin staining in the endothelium or the smooth muscle. In WT-treated vessels, there was minimal hypereosinophilic staining in the smooth muscle, possibly representing mild hypoxic changes. There was no necrotic debris, loss of nuclei or loss of structure visualized. The endothelium and elastic lamina of WT-treated vessels appeared normal.

### Example 4

### Evaluation of Endothelial Cell and Smooth Muscle Cell Apoptosis

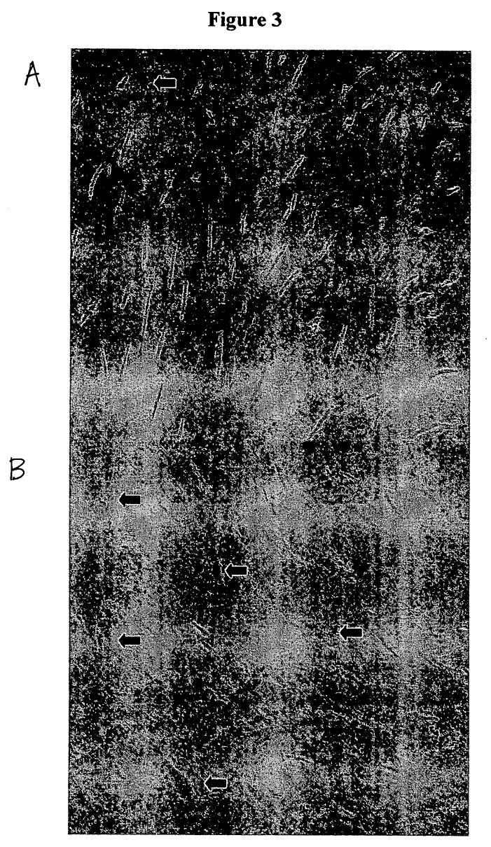

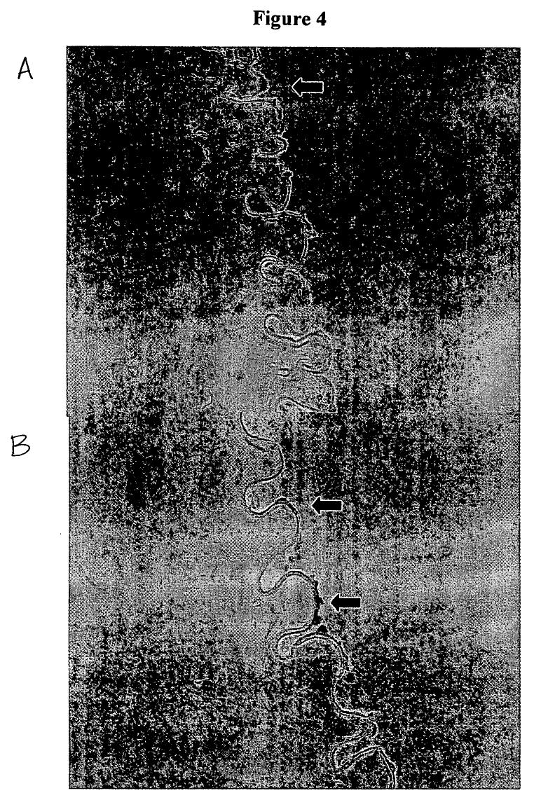

Forty-eight hours after treatment with WT and incubation in culture medium, the vessels were examined for apoptosis in the smooth muscle of WT-treated vessels, there was a greater percentage of TUNEL-positive cells (4.63±1.04%) than in control vessels (0.35±0.16%, P=0.003) (FIG. 3). In the endothelium, there were relatively more TUNEL-positive cells in the WT group (16.88%±4.90%) than in the control group (4.47±2.90%). This difference, however, did not reach statistical significance (P=0.058) (FIG. 4).

The present disclosure introduces the concept that irreversibly inhibiting MLCK activity, inhibiting rho-rho kinase pathway or promoting myosin light chain phosphatase activity attenuates brachial artery contractile activity in response to a broad spectrum of vasoconstrictor agents. Moreover, for the first time, the present disclosure demonstrates that this brief treatment reduces contractile responses by 90% at 2 h and 50% at 48 h following washout of the MLCK inhibitor. Therefore, this treatment would not only prevent intraoperative vasospasm caused by harvesting trauma and manipulation, but also would obviate the need for the initial postoperative use of calcium channel blockers and nitrates and avert their potential complications. Inhibiting vasospasm in the early postoperative period is critical since during this time, the patient has high levels of circulating catecholamines and other endogenous vasoconstrictors (i.e. endothelin and thromboxane).

In this disclosure, the effects of wortmannin-treated vs. vehicle-treated vessels are compared. However, using a similar protocol, the effects of phenoxybenzamine, an irreversible α-1 receptor blocker [Corvera et al., 2003; Dipp et al. (2001) Phenoxybenzamine is more effective and less harmful than papaverine in the prevention of radial artery vasospasm, Eur. J. Cardiothorac. Surg., 19:482-6; Velez et al., 2001] and papaverine were investigated on radial artery contraction both at 2 and 48 h after treatment [Velez et al., 2001]. In a previous study, phenoxybenzamine was effective in preventing alpha-agonist-induced contractions at 48 h following treatment; however, while phenoxybenzamine provides long-lasting inhibition of alpha-adrenergic vasoconstrictors, it does not prevent vasospasm induced by other potentially important stimuli [Conant et al. (2003) Phenoxybenzamine treatment is insufficient to prevent spasm in the radial artery: the effect of other vasodilators, J. Thorac. Cardiovasc. Surg., 126:448-54]. It has also previously been demonstrated that papaverine, the current gold standard prophylactic treatment for intraoperative vasospasm, had a brief duration of action, with virtually no effect after the drug has been washed away [Velez et al., 2001]. In contrast, the results from the current study indicate that wortmannin significantly attenuates contraction induced by both calcium-dependent and independent mechanisms at 48 h following treatment and washout.

Nitric oxide donors and calcium channel blockers are commonly used agents to treat postoperative vasospasm. Like papaverine, these vasodilators are short-acting and are usually administered as an intravenous infusion during the first 24 postoperative hours, followed by oral administration. As a result of this systemic administration, they may have undesirable side effects such as hypotension, brady-cardia, and negative inotropy [Acar et al., 1992; Shapira et al. (2000) Nitroglycerine is preferable to diltiazem for prevention of coronary bypass conduit spasm, Ann. Thorac. Surg., 70:883-9]. In addition, prolonged use of nitroglycerin has been associated with endothelial dysfunction and nitrate tolerance, which may cause reflex vasospasm upon withdrawal [Schulz et al. (2002) Functional and biochemical analysis of endothelial (Dys)function and NO/cGMP signaling in human blood vessels with and without nitroglycerine pretreatment, Circulation, 105:1170-5]. Therefore, treatment of postoperative vasospasm with these presently administered agents may be of limited effectiveness.

Although direct MLCK inhibition prevents long-term vasospasm in the current study, the use of wortmannin as the specific inhibitor has some limitations, particularly the observation of smooth muscle and endothelial cell apoptosis. This finding can be explained by the fact that wortmannin, in addition to inhibiting MLCK, is a potent inhibitor of phosphoinositide-3 (PI-3) kinase [Wymann et al. (1996) Wortmannin inactivates phosphoinositide 3-kinase by covalent modification of Lys-802, a residue involved in the phosphate transfer reaction, Mol. Cell Biol., 16:1722-33], which has been shown to have anti-apoptotic activity in some cell culture lines. Therefore, inhibition of the anti-apoptotic effects of PI-3 kinase may allow initiation of apoptosis triggered by other signals. Accordingly, blockade of PI-3 kinase activity with wortmannin has been associated with the development of apoptosis in these cells in vitro [Yao et al. (1995) Requirement for phosphatidylinositol-3 kinase in the prevention of apoptosis by nerve growth factor, Science, 267:2003-6]. It is therefore likely that the smooth muscle and endothelial cell apoptosis seen in our study is a result of PI-3 kinase inhibition by wortmannin.

While apoptosis of the smooth muscle and endothelium is undesirable, its implications for long-term graft function and patency are unclear. Other antispasmodic treatments, including papaverine have been shown to have similar pro-apoptotic effects on vessels. Several studies have shown that papaverine causes both impairment of endothelial function and endothelial cell apoptosis in radial and internal mammary arteries [Dipp et al., 2001; Gao et al., 2003; Rubens et al. (1998) Papaverine solutions cause loss of viability of endothelial cells, J. Cardiovasc. Surg. 39:193-9]. Nevertheless, topical application of papaverine has not proven to have any long-term detrimental effects. With regard to the current study, inhibition of PI-3 kinase and its physiological consequences could potentially be averted by using a more selective MLCK inhibitor which is devoid of PI-3 kinase inhibitory activity. While such agents have been described in the literature, they are not currently commercially available [Yano et al. (1995) Biochemical and pharmacological studies with KT7692 and LY294002 on the role of phosphatidylinositol 3-kinase in FcERI-mediated signal, Biochem. J., 312:145-150]. In many ex vivo organ chamber studies vascular endothelial function is tested by measuring relaxation responses to an endothelial-dependent vasodilating agent, such as acetylcholine or bradykinin. However, vasorelaxation induced by these agonists depends on the phosphorylation and activation of eNOS by Akt, a PI-3 kinase-dependent protein kinase [Kitayama et al., 2000]. Therefore, since wortmannin inhibits PI-3 kinase, and consequently phosphorylation of Akt into its active form, it follows that acetylcholine-dependent vasodilation would also be inhibited by wortmannin. In this study, endothelial-dependent relaxation is utilized as an assessment of endothelial function. In addition, since inhibition of MLCK with wortmannin limits vasoconstriction almost completely in the early period and by 50% at 48 h, relaxation responses could not be accurately compared to control groups, which contracted to a greater extent. Therefore, histology and TUNEL staining were used to evaluate potential cellular damages caused by treatment with wortmannin. Despite the potential detrimental effects of PI-3 kinase inhibition outlined above, a recent study by Su et al. [Su et al. (2004) Phosphatidylinositol 3-kinase modulates vascular smooth muscle contraction by calcium and myosin light chain phosphorylation-independent and -dependent pathways, Am. J. Physiol. (Heart Circ. Physiol.), 286:H657-H666] suggests that the inhibition of PI-3 kinase may indeed be an important mechanism in the antispasmodic action of wortmannin. While the study did not examine the effects of wortmannin specifically, the results using a selective PI-3 kinase inhibitor suggest trial the activation of PI-3 kinase may be involved in MLC-dependent and independent pathways of vascular smooth muscle contraction. Therefore, while the inhibitory effect of wortmannin on PI-3 kinase may have some undesirable consequences, it may also be partly responsible for the potent antispasmodic effect presented in this study.

Finally, wortmannin has been described in previous literature as an irreversible inhibitor of MLCK [Wymann et al., 1996]. However, the results of the present study indicate that 48 h after treatment with wortmannin, the vessels regained about 50% of their contractile activity. Why this occurs is not entirely clear, but may be explained in part by the turnover and new synthesis of MLCK. Alternatively, there may be unidentified MLCK-independent mechanisms of smooth muscle cell contraction which are not active initially, but become activated at the 48 h time point. Furthermore, it is possible that in a biological setting, physiologically circulating factors might produce vascular changes that reverse the inhibitory properties of the drug. Although the physiologic milieu can be mimicked by storing the treated vessels in culture medium at physiologic pH and temperature, the long-term inhibitory properties of wortmannin would ideally be assessed in an in vivo setting.

In summary, irreversible inhibition of MLCK by wortmannin nearly abolishes vasoconstriction stimulated by an array of vasopressor agents in the early period after pre-treatment. Furthermore, this treatment strategy provides sustained attenuation of vasoconstriction in response to all potential vasoconstrictor stimuli for up to 48 h.

The examples are given to illustrate the disclosure and are not intended to limit the claims of the disclosure in any manner. One skilled in the art will appreciate from reading this disclosure that various changes in form and detail can be made without departing from the true scope of the disclosure.

Therefore, the following is claimed:

1. A method for preventing or reducing vasospasms in a host comprising administering to the host an amount of an MLCK inhibitor sufficient to prevent vasospasms.

2. The method of claim 1, wherein the MLCK inhibitor prevents vasospasm by about 90% for at least about 1 to about 2 hours post administration.

3. The method of claim 1, wherein the MLCK inhibitor prevents vasospasm by about 50% for at least about 1 to about 48 hours post administration.

4. The method of claim 1, wherein the MLCK inhibitor comprises wortmannin, a derivative of wortmannin, a pharmaceutically acceptable salt thereof, or a prodrug thereof.

5. The method of claim 1, wherein the MLCK inhibitor reversibly or irreversibly inhibits myosin light chain kinase.

6. The method of claim 1, wherein the MLCK inhibitor is administered non-systemically.

7. The method of claim 1, wherein the host comprises a mammal.

8. The method of claim 7, wherein the host is selected from the group consisting of a human, primate, ungulate, swine, dog, cat, rodent, and rabbit.

9. The method of claim 1, wherein a radial artery, internal mammary artery, saphenous vein graft, thoracic aorta, basilar artery, gastroepiploic artery, mesenteric arteries or carotid artery are prevented from contracting.

10. The method of claim 5, wherein the irreversible MLCK inhibitor comprises 5′flourosulfonylbenzoyl adenosine (FSBA), a derivative thereof, a fragment thereof, a pharmaceutically acceptable salt thereof, or a prodrug thereof.

11. The method of claim 1, wherein the MLCK inhibitor inhibits vasospasms caused by a contraction that is calcium independent.

12. The method of claim 1, wherein the vasospasm is caused by a contraction that is calcium dependent.

13. A composition comprising an inhibitor of MLCK in an amount effective to reduce contractile responses of vascular smooth muscle in a host by about 90% for at least about 20 minutes post administration.

14. The composition of claim 13, wherein the MLCK inhibitor is in an amount effective to reduce contractile responses of vascular smooth muscle by about 90% for at least about 1 to 2 hours post administration.

15. A composition of claim 13, wherein the MLCK inhibitor is in an amount effective to reduce contractile responses of vascular smooth muscle in a host by about 50% for at least about 1 to about 48 hours post administration.

16. A composition of claim 13, wherein the MLCK inhibitor comprises wortmannin, a derivative of wortmannin, a pharmaceutically acceptable salt thereof, or a prodrug thereof

17. A method for identifying a test compound for preventing or reducing vasospasms comprising:
(a) contacting vascular smooth muscle cells with the test compound;
(b) assaying the cells for contraction; and
(c) selecting the compound that reduces or prevents vascular smooth muscle cell contraction and inhibits MLCK activity.

18. The method of claim 17, further comprising the steps of determining apoptotic effects of the test compound and selecting the test compound with reduced apoptotic effects compared to wortmannin.

19. The method of claim 17, wherein the test compound has less than about 50% to about less than about 10% inhibition of another enzyme.

20. The method of claim 19, wherein the other enzyme is PI-3 kinase.

21. The method of claim 17, wherein the test compound irreversibly inhibits MLCK.

22. The method of claim 17, wherein the vascular smooth muscle cells are contacted with the test compound in vitro or in vivo.

23. A method for preventing vasospasm in a host comprising administering an irreversible inhibitor of MLCK to a vessel at a site of a graft in an amount sufficient to inhibit or reduce contraction of the vessel at the site of the graft.

24. The method of claim 23, wherein the composition comprises wortmannin, a derivative thereof, a fragment thereof, or a prodrug thereof.

25. The method of claim 23, wherein the MLCK inhibitor prevents vasospasm by about 90% for at least about 1 to about 2 hours post administration.

26. The method of claim 23, wherein the MLCK inhibitor prevents vasospasm by about 50% for at least about 1 to about 48 hours post administration.


SEQUENCE LISTING (1 sequence)

SEQ ID NO: 1 - protein, 9 aa, Artificial Sequence
  Synthetic peptide (MLCK Inhibitor)
RKKYKYRRK